(19) United States
(12) Patent Application Publication
(10) Pub. No.: US 2026/0248683 A1
(43) Pub. Date: Aug. 27, 2026

(54) AUTOMATED TOURNIQUET AND REAL-TIME HEMODYNAMIC FEEDBACK DEVICE AND METHODS OF USING THE SAME FOR IMPROVING CPR OUTCOMES

(71) Applicant: TuroLife LLC, Bala Cynwyd, PA (US)

(72) Inventor: Justin Joffe, Pikesville, MD (US)

(73) Assignee: TuroLife LLC, Bala Cynwyd, PA (US)

(21) Appl. No.: 19/651,018

(22) Filed: Apr. 17, 2026

Related U.S. Application Data
(63) Continuation of application No. PCT/US2025/056392, filed on Nov. 20, 2025.
(60) Provisional application No. 63/723,320, filed on Nov. 21, 2024.

(51) Int. Cl. A61H 31/00; A61B 17/00; A61B 17/132; A61B 17/135; A61B 90/00

(52) U.S. Cl. CPC A61H 31/006; A61B 17/1322; A61B 17/1355; A61B 90/03; A61B 90/06; A61H 31/005; A61B 2017/00022; A61B 2017/00199; A61B 2017/00221; A61B 2017/00544; A61B 2017/00557; A61B 2090/064; A61H 2201/0103; A61H 2201/018; A61H 2201/1238; A61H 2201/501; A61H 2201/5043; A61H 2201/5097; A61H 2203/0456; A61H 2205/06; A61H 2205/084; A61H 2205/10; A61H 2230/255; A61H 2230/305

(57) ABSTRACT

Disclosed are systems and related methods for augmenting blood supply to vital organs in a patient. An optional system includes a plurality of limb cuffs. Each limb cuff has a real-time hemodynamic sensor and an inflatable sleeve configured to be placed around a limb of the patient to autonomously perform occlusion of the limb in response to real-time hemodynamic readings generated by the real-time hemodynamic sensor. An optional method includes placing one limb cuff on each separate limb of the patient and allowing each limb cuff to autonomously perform a limb occlusion operation and to adjust a limb occlusion setting in response to the real time hemodynamic readings so as to augment blood supply to the vital organs, optionally while performing CPR on the patient.

### CROSS REFERENCE TO RELATED APPLICATIONS

This application claims priority to U.S. Provisional Patent Application No. 63/723,320, entitled AUTOMATED TOURNIQUET-BLOOD PRESSURE HYBRID DEVICE FOR OPTIMIZING CPR OUTCOMES and filed on Nov. 21, 2024. The aforementioned priority application is hereby incorporated by reference herein in its entirety.

### FIELD OF THE INVENTION

The presently disclosed technology relates generally to devices, systems and methods for augmenting blood supply to vital organs in a patient, for example during cardiopulmonary resuscitation (CPR). Such devices, systems and methods comprise a plurality of limb cuffs, each of which includes a real-time hemodynamic sensor and an inflatable sleeve that autonomously performs limb occlusion in response to real-time hemodynamic readings from the sensor.

### BACKGROUND OF THE INVENTION

Cardiac arrest remains one of the leading causes of mortality worldwide, particularly in out-of-hospital settings. The physiologic objective of cardiopulmonary resuscitation (CPR) is to preserve cerebral and coronary perfusion until return of spontaneous circulation (ROSC) is achieved. However, conventional CPR techniques provide only a fraction of normal cardiac output and often fail to maintain sufficient central perfusion.

Tourniquet-Assisted CPR (T-CPR) is a concept supported by emerging preclinical studies demonstrating that peripheral vascular occlusion can increase systemic vascular resistance (SVR), redirect blood flow centrally, and improve perfusion pressures. Existing tourniquet technologies, however, lack dynamic, real-time control and are not designed for CPR contexts. Moreover, prior attempts at automation have relied on centralized pneumatic manifolds (e.g., Heartbeat's SAVER system), which suffer from single-point failure risk and lack autonomous limb-level control.

Thus, there exists a need for a fully autonomous, limb-specific, closed-loop tourniquet system capable of dynamically adjusting occlusion pressure in real time during CPR, without reliance on centralized air distribution systems or primary manual intervention.

### SUMMARY OF THE INVENTION

This need, and others, are met by the presently disclosed technology.

Unlike prior systems, which depend on shared pneumatic infrastructure or require human operation, the presently disclosed technology introduces what Applicant refers to as an Automated Air-Cuff Tourniquet (AAT) platform—a system including a set of independent, preferably battery-operated devices (limb cuffs) applied to each extremity, each preferably equipped with its own microcontroller, air pump, solenoid valve, pressure transducer, and motion sensor. These devices are preferably configured to operate autonomously to determine and maintain minimal limb occlusion pressure (MLOP), compensate for CPR-induced artifacts, and enable real-time waveform analysis with gradual pressure titration upon ROSC detection to preserve systemic vascular resistance (SVR), promote hemodynamic stability, and optimize post-resuscitative neurological outcomes and survival to hospital discharge.

Furthermore, the disclosed technology optionally integrates wireless data telemetry to an optional central interface for clinical feedback without compromising device independence. This decentralized architecture avoids single-point failure modes, ensures redundancy across limbs, and allows selective activation in tactical or resource-limited environments (e.g., military, EMS, disaster response).

Importantly, the disclosed technology is purpose-built for the physiologic conditions of CPR, incorporating algorithms specifically tuned for hemodynamic variability, chest compression artifacts, and low-flow circulatory states. This level of precision and adaptability is a distinct advance over prior devices, which focus only on static limb compression or non-CPR contexts.

Therefore, the disclosed technology fills a critical clinical and technological gap by offering a deployable, intelligent, extremity-based tourniquet system designed to enhance central perfusion during cardiac arrest, with the potential to significantly improve survival and neurologic outcomes.

The AAT system is not necessarily intended to replace or modify current American Heart Association (AHA) guidelines (or other similar guidelines respective to AAT geographic deployment) for CPR. Rather, it is designed to function as an adjunctive hemodynamic support tool that operates in parallel with existing resuscitative protocols. AAT is purposefully engineered to augment perfusion dynamics during CPR, preferably without obstructing or delaying established clinical interventions such as intraosseous (IO) access, intravenous (IV) placement, or central venous catheterization. The limb-specific design permits high-and-tight placement on all four extremities while still preserving access points commonly used for vascular cannulation—including the anterior tibia, humeral head, antecubital fossa, and femoral or internal jugular veins. If vascular access is required in a limb where an AAT device is deployed, the cuff can be rapidly repositioned or temporarily deflated without interrupting CPR or compromising the system's integrity. In this way, AAT is compatible with and complementary to current advanced life support protocols, providing additional circulatory support without interfering with standard-of-care resuscitation procedures.

In one optional aspect, the disclosed concept is directed to a method for augmenting blood supply to vital organs in a patient. The method includes providing a system having a plurality of limb cuffs. Each limb cuff includes a real-time hemodynamic sensor and an inflatable sleeve configured to be placed around a limb of the patient to autonomously perform occlusion of the limb in response to real-time hemodynamic readings generated by the real-time hemodynamic sensor. The method further includes placing one limb cuff of the plurality of limb cuffs on each separate limb of the patient and allowing each limb cuff to autonomously perform a limb occlusion operation and to adjust a limb occlusion setting in response to the real time hemodynamic readings generated by the real-time hemodynamic sensor so as to augment blood supply to the vital organs.

Optionally, in any embodiment, the inflatable sleeve has an inner surface configured to contact a respective limb while in use and an outer surface. Each limb cuff includes a housing that is more rigid than the inflatable sleeve. The housing features an inner wall having a fixed diameter that defines an outer boundary beyond which the inflatable sleeve cannot expand.

Optionally, in any embodiment, the housing has a bottom portion that is symmetrical and shaped in a manner that prevents rolling or rotation of the housing relative to a substantially flat surface on which the bottom portion is placed, when the patient is lying in a supine position on the substantially flat surface and while the limb cuff is placed around a limb of the patient.

Optionally, the plurality of limb cuffs consists of the following four limb cuffs: (a) a right arm limb cuff; (b) a left arm limb cuff; (c) a right leg limb cuff; and (d) a left leg limb cuff.

Optionally, in any embodiment, the system includes a user interface with a monitor to provide real time system status and patient hemodynamic feedback. The user interface is physically separate from the plurality of limb cuffs.

Optionally, in any embodiment, there are no physical connections between the plurality of limb cuffs. For example, there are no wires or tubes connecting the plurality of limb cuffs.

Optionally, in any embodiment, each limb cuff includes a microcontroller for providing autonomous operation of the real-time hemodynamic sensor and the inflatable sleeve.

Optionally, in any embodiment, each limb cuff includes: (a) an air pump in fluid connection with the inflatable sleeve to inflate the inflatable sleeve; (b)a solenoid valve in fluid connection with the inflatable sleeve to release air from the inflatable sleeve when the solenoid valve is open; (c) a power source, optionally a battery, to provide electrical power to each limb cuff; and (d) optionally an accelerometer configured to cancel out external movement and noise to ensure accurate real-time hemodynamic readings.

Optionally, in any embodiment, each limb cuff includes a wireless communication interface for remote monitoring and feedback. Optionally, the wireless communication interface transmits data via Bluetooth Low Energy (BLE), WiFi or Ultra Wideband to a user interface.

Optionally, in any embodiment, the real-time hemodynamic sensor is configured to measure flow according to the following algorithm: flow=change in pressure divided by vascular resistance.

Optionally, in any embodiment, each limb cuff operates physically and electronically independently of every other limb cuff of the plurality of limb cuffs.

Optionally, in any embodiment, the real-time hemodynamic sensor includes two of the same type of sensor, a first of which is configured for measuring minimal limb occlusion pressure (MLOP) and a second of which is configured for proximal arterial waveform detection.

Optionally, the method includes performing cardiopulmonary resuscitation (CPR) on a patient by augmenting blood supply to vital organs in the patient according to any of the methods described herein, while simultaneously applying chest compressions to the patient. Optionally, the system operates to increase blood supply to the vital organs during CPR.

Optionally, in any embodiment, the system determines minimal limb occlusion pressure (MLOP, optionally in mm Hg) of the patient optionally using oscillometric feedback and also determines blood flow at an occlusion site using hemodynamic feedback. A limb occlusion setting is automatically adjusted in real time in response to MLOP and blood flow measurements.

Optionally, in any embodiment, the plurality of limb cuffs consists of two lower extremity cuffs and two upper extremity cuffs. Optionally, each of the two lower extremity cuffs are placed on a respective leg of the patient and activated, after which each of the two upper extremity cuffs are placed on a respective arm of the patient and activated.

Optionally, when performing CPR according to any of the methods disclosed herein, upon return of spontaneous circulation (ROSC) in the patient, an ROSC mode is initiated in the system. In ROSC mode, the plurality of limb cuffs gradually reduce occlusion pressure in order to maintain stable systemic vascular resistance (SVR) and thereby mitigate a risk of acute central hypotension.

Optionally, when performing CPR according to any of the methods disclosed herein, the method is capable of being performed while an automated mechanical CPR device applies the chest compressions to the patient.

Optionally, in any embodiment, the method does not increase intrathoracic pressure.

Optionally, the system according to the disclosed technology is configured to provide real-time hemodynamic feedback during CPR by using one or more specialized sensors placed near the site of limb occlusion. Unlike traditional tourniquet or blood pressure cuffs that measure only static pressure, the present system incorporates proximal arterial waveform detection to actively read the dynamic blood pulse near the cuff. Two sensors are preferably used: one determines the MLOP required to stop arterial flow, and the second monitors the actual arterial pressure waveform pushing against the cuff. This dual-sensor approach enables the device to evaluate the quality of CPR in real time, such as compression depth and rate, by detecting oscillations generated by chest compressions. In use, the system increases SVR by occluding the limbs, which redirects blood flow centrally toward the heart, brain, and lungs. As the patient's condition changes—for example, if ROSC occurs—the system can gradually reduce pressure through a ROSC-controlled release mode to maintain perfusion while preventing sudden blood redistribution. Unlike central circulatory adjuncts such as Resuscitative Endovascular Balloon Occlusion of the Aorta (REBOA) or load-distributing band devices that increase intrathoracic or intra-aortic pressure, the disclosed technology achieves systemic vascular resistance (SVR) modulation peripherally, without compromising thoracic dynamics or impeding venous return.

### BRIEF DESCRIPTION OF THE DRAWINGS

A full understanding of the invention can be gained from the following description of the preferred embodiments when read in conjunction with the accompanying drawings in which:

FIG. 1 is a top plan view of a supine patient with a limb cuff according to an optional aspect of the disclosed concept on each one of the patient's appendages;

FIG. 2 is an isometric exploded view of one of the limb cuffs that is used on one of the patient's arms in FIG. 1, the limb cuff including a cuff housing and an inflatable sleeve assembled within the housing;

FIG. 3 is another isometric exploded view of the limb cuff of FIG. 2;

FIG. 4 is an isometric view of the limb cuff of FIGS. 2 and 3 in assembled form;

FIG. 5 is a schematic view of the limb cuff of FIG. 4 disassembled to illustrate internal components;

FIG. 6A is a partial section view of the limb cuff of FIGS. 2-5 along a central section plane while the limb cuff is placed around the patient's arm with the inflatable sleeve in a pre-inflated state;

FIG. 6B is a partial section view of the limb cuff as shown in FIG. 6A, except with the inflatable sleeve in an inflated state to carry out an occlusion operation.

FIG. 7 is a plan view of a monitor display of an exemplary user interface that may be included in connection with systems and methods using limb cuffs according to the disclosed concept;

FIG. 8 illustrates the supine patient of FIG. 1 having CPR performed on him while a limb cuff is on each one of his arms and legs.

FIG. 9 is a top plan view of the limb cuff of FIGS. 2-6B having an optional power switch and LCD screen for providing patient status feedback and functional modes of the limb cuff.

### DETAILED DESCRIPTION OF THE INVENTION

While systems, devices and methods are described herein by way of examples and embodiments, those skilled in the art recognize that the presently disclosed technology is not limited to the embodiments or drawings described. Rather, the presently disclosed technology covers all modifications, equivalents and alternatives falling within the spirit and scope of the appended claims.

Any headings used herein are for organizational purposes only and are not meant to limit the scope of the description or the claims. As used herein, the word “may” is used in a permissive sense (i.e., meaning having the potential to) rather than the mandatory sense (i.e., meaning must). Unless specifically set forth herein, the terms “a,” “an” and “the” are not limited to one element but instead should be read as meaning “at least one.” The terminology includes the words noted above, derivatives thereof and words of similar import.

One or more features of any particular embodiment can be omitted or included in (e.g., added to) another embodiment, each of which form part of the presently disclosed technology.

### Systems and Devices of the Disclosed Technology

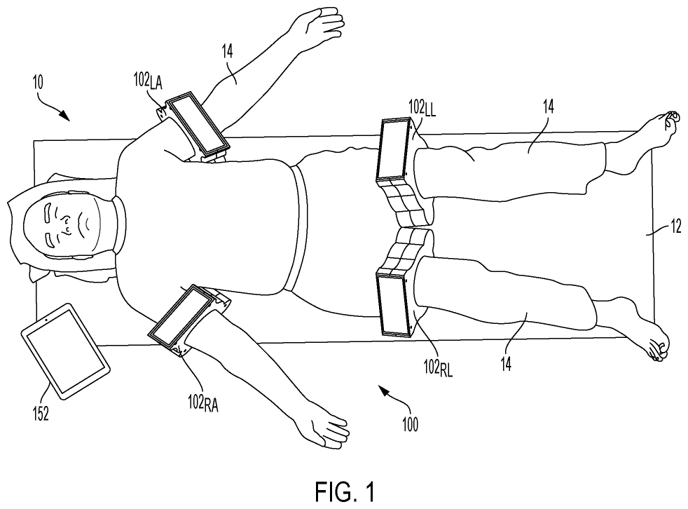

Referring now in detail to the various figures, wherein like reference numerals refer to like parts throughout, there is shown in FIG. 1 a patient 10 in a supine position on a substantially flat surface 12, e.g., the ground, a bed or a gurney. The patient is in medical distress and is in need of acute treatment due to cardiopulmonary arrest, a sudden medically destabilizing drop in blood pressure and/or hemorrhage. The patient 10, as shown, is undergoing or is about to undergo treatment to achieve medical stability. Such treatment would include use of a system 100 according to an optional aspect of the disclosed technology. The system 100 is used to augment blood supply to vital organs in the patient 10.

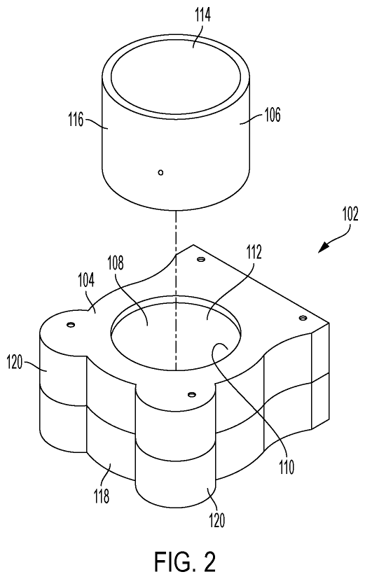

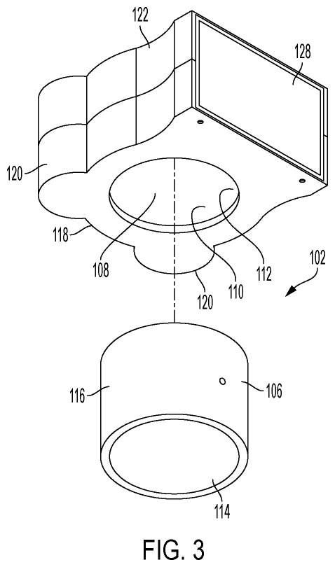

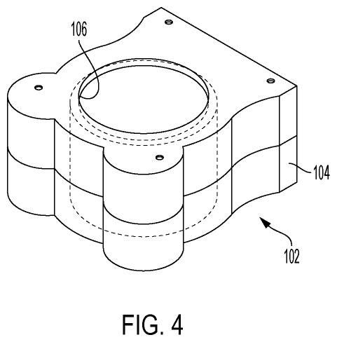

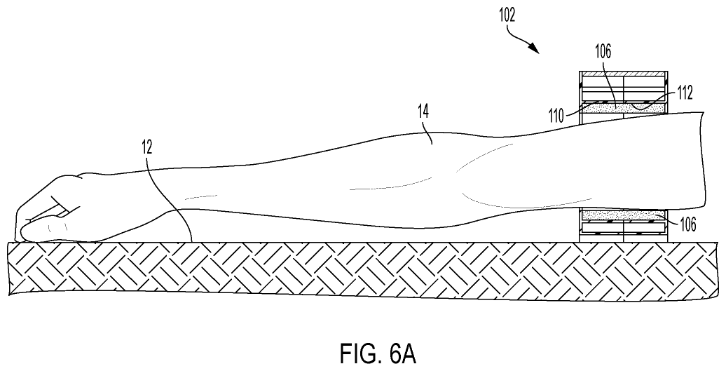

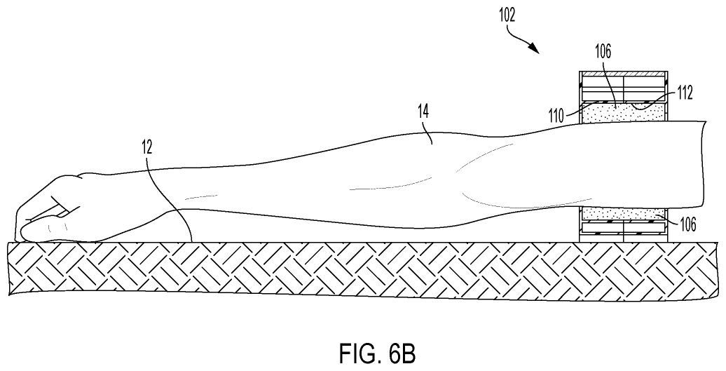

Various optional aspects of the system 100 are illustrated in FIGS. 1-9. The system 100 includes a plurality of limb cuffs 102. Optionally, the system 100 includes four limb cuffs 102, namely a right arm limb cuff 102_{RA}, a left arm limb cuff 102_{LA}, a right leg limb cuff 102_{RL} and a left leg limb cuff 102_{LL}. Each limb cuff 102 preferably includes a housing 104 and an inflatable sleeve 106 disposed axially within a central aperture 108 in the housing 104. The housing 104 is more rigid than the inflatable sleeve 106. The housing 104 comprises an inner wall 110 defining the central aperture 108 and having a fixed diameter at least when it is ready for use. Optionally (not shown), the inner wall of the housing is adjustable to fit different arm sizes, but once adjusted for use, has the aforementioned fixed diameter. The inner wall 110 defines an outer boundary 112 beyond which the inflatable sleeve 106 cannot expand when in use. Each inflatable sleeve 106 has an inner surface 114 configured to contact the patient's limb while in use, i.e., placed on the patient's limb and operational. By “placed on the patient's limb” or “placed around the patient's limb”, it is meant that at least one of the patient's arms or at least one of the patient's legs protrudes axially through the inflatable sleeve 106, e.g., as shown in FIGS. 6A and 6B. FIG. 6A is a partial section view of the limb cuff 102 along a side central section plane while the limb cuff is placed around the patient's arm with the inflatable sleeve 106 in a pre-inflated state. FIG. 6B is the same view, except with the inflatable sleeve 106 in an inflated state to carry out an occlusion operation. It should be noted that in the inflated state, the inflatable sleeve 106 does not expand beyond the outer boundary 112 defined by the inner wall 110. Each inflatable sleeve 106 also includes an outer surface 116 facing (optionally contacting) the inner wall 110.

The inflatable sleeve 106 may at least in part be made from materials from which traditional blood pressure cuffs are made. Optionally, the inflatable sleeve 106 has an outer shell of polyurethane-coated nylon, thermoplastic polyurethane (TPU), or medical grade-silicone elastomer. The inflatable sleeve 106 may include an inner bladder formed of medical-grade silicone or TPU. The inflatable sleeve 106 may also include padding from closed-cell antimicrobial polyurethane foam to help ensure patient safety in use and reduce risk of damage to a patient's limb during prolonged and/or repeated occlusion operations.

The housing 104 optionally includes a bottom portion 118 that is preferably symmetrical and shaped in a manner that prevents rolling or rotation of the housing 104 relative to the substantially flat surface 12 on which the bottom portion 118 rests when the limb cuff 102 is in use. For example, the bottom portion 118 of the housing 104 includes opposing rounded legs 120 that help to firmly seat the housing 104 on the substantially flat surface 12 to keep the housing 104 in place. This functional feature is helpful to ensure that certain sensors in the limb cuffs 102 are properly positioned adjacent to portions of the anatomy from which the sensors are configured to generate important readings, as discussed below. The housing 104 of the right arm limb cuff 102_{RA} and left arm limb cuff 102_{LA} preferably includes a concave curved medial surface 122 configured to approximately follow and fit snugly against the curvature of the lateral part of the patient's torso adjacent the arm about which the limb cuff 102 is placed. Optionally, the medial surfaces of the housings 104 of the right leg limb cuff 102_{RL} and the left leg limb cuff 102_{LL}, i.e., the walls of the housings 104 between both legs, could be squared off and thus have substantially flat vertical surfaces (not shown). This optional feature may allow the housings 104 for the leg limb cuffs 102 to be flush with each other if they are in contact during use, improving their positional stability. Such configuration may also help save space on a narrow surface (e.g., gurney) and make transport easier.

As discussed in more detail below, each limb cuff 102 comprises a real-time hemodynamic sensor. The inflatable sleeve 106 is configured to be placed around a respective limb 14 of the patient 10 to autonomously perform occlusion of the limb 14 in response to real-time hemodynamic readings generated by the real-time hemodynamic sensor so as to augment blood supply to the patient's vital organs. When a limb cuff 102 is placed on each limb 14, e.g., as shown in FIG. 1, the occlusion that each limb cuff 102 performs helps promote perfusion of blood into the patient's vital organs when the patient is medically unstable, e.g., due to cardiac arrest or a sudden medically destabilizing drop in blood pressure.

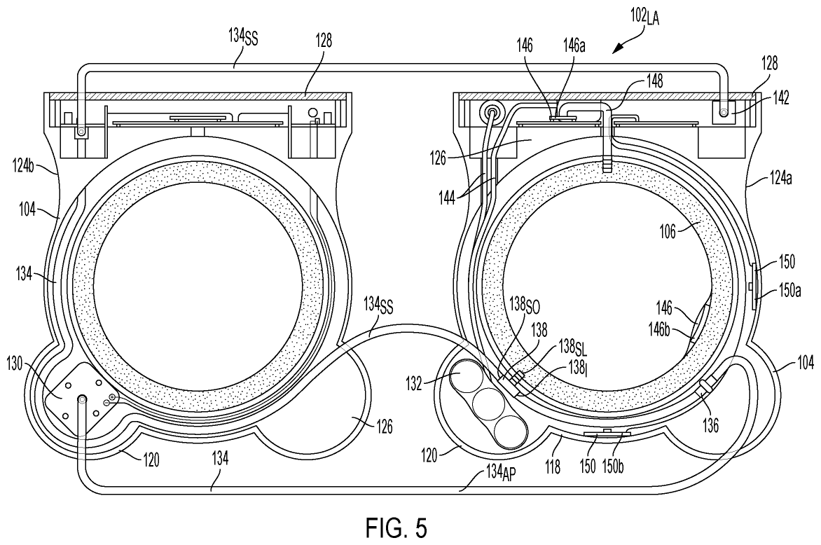

Optionally, as shown in FIG. 5, the housing 104 comprises a first housing shell 124a assembled to a second housing shell 124b. When assembled, the first housing shell 124a and second housing shell 124b form a cavity 126 within which various components and electronics may be encased. Optionally, the housing 104 is made from a polymer with metal (e.g., stainless steel) reinforcement. For example, the housing 104 may be made from polycarbonate, thermoplastic polyurethane, or acrylonitrile butadiene styrene, optionally with steel reinforcement. In alternative embodiments (not shown), the housing may include a single primary receptacle with a cover, rather than two separate shell halves. In any embodiment, including that illustrated and described herein, the housing 104 should be relatively rigid and durable. The various embodiments of the housing described herein are all within the scope of the disclosed concept and are merely exemplary.

FIG. 5 shows an optional embodiment of a left arm limb cuff 102_{LA} in a schematic view in which various internal components are shown when the first housing shell 124a and second housing shell 124b are disassembled. This figure shows a cross section of the inflatable sleeve 106 and an LCD screen 128 at the top of the limb cuff 102; however the remainder of the limb cuff 102 is shown schematically, rather than as a cross-section, to best illustrate various internal components. The respective positions and locations of the various components within the housing 104 are merely exemplary and nonlimiting.

The cavity 126 includes an air pump 130 and a battery 132 or other power source. Optionally the air pump 130 is located within one rounded leg 120 in the bottom portion 118 of the housing 104 and the battery 132 is located within the other rounded leg 120 in the bottom portion 118 of the housing 104. Optionally, the battery 132 is a rechargeable 12V lithium-ion battery. Optionally, the air pump 130 is a miniature DC (6V or 12V) air pump capable of generating at least 200 mm Hg. The air pump 130 is operably connected to tubing 134 through which pressurized air would flow when the air pump 130 is actuated. The tubing 134, in an air pump section 134_{AP} thereof, extends from the air pump 130, optionally to/through a one-way valve 136 (to prevent reverse flow of air from that point) and then to a three-way fitting 138 (optionally barbed fitting). The three-way fitting 138 includes an inlet 138_{I} that receives the pumped air, a sleeve outlet 138_{SL} that directs pumped air into a port in the inflatable sleeve 106 and a solenoid section outlet 138_{SO}. The solenoid section outlet 138_{SO} of the tubing 134 is connected to a solenoid section 134_{SS} of the tubing 134 that extends through the cavity 126 and ultimately to a solenoid valve 142 that is optionally located in an upper portion of the housing 104, beneath the LCD screen 128. When an occlusion operation is performed, the air pump 130 is actuated to direct air through the air pump section 134_{AP} of the tubing 134, to inflate the inflatable sleeve 106. When the inflatable sleeve 106 is to be deflated, the solenoid valve 142, which is normally closed in its rest state, is triggered to open, allowing air to flow from the inflatable sleeve 106, into the three-way fitting 138, through the solenoid section 134_{SS} of the tubing 134 and out from the limb cuff 102. Preferably these operations are all effectuated autonomously.

The battery 132 is connected to wiring 144 that extends to various electronic components, some of which are optionally stored beneath the LCD screen 128. One such component is the real-time hemodynamic sensor 146. Optionally, the real-time hemodynamic sensor 146 actually comprises more than one sensor. For example, in the embodiment shown, the real-time hemodynamic sensor 146 comprises two of the same type of sensor (i.e., the same type of hardware). However, they are programmed differently. A first sensor 146a of the real-time hemodynamic sensor 146 is configured for measuring minimal limb occlusion pressure (MLOP) and a second sensor 146b of the hemodynamic sensor is configured for proximal arterial waveform detection. For example, the first sensor 146a and second sensor 146b may both be MPX5010DP, BMP388, or MPXV7002DP sensors. The first sensor 146a is in fluid communication with the inflatable sleeve 106 via a short sensor tube 148. As an alternative (not shown) that is within the scope of the disclosed technology, the real-time hemodynamic sensor consists of one single sensor that is configured for both proximal arterial waveform detection and measuring MLOP.

In any embodiment, the real-time hemodynamic sensor 146 that is configured for proximal arterial waveform detection (as shown, the second sensor 146b) is to be located in or on the inflatable sleeve 106 at a position that is configured to be adjacent to the primary artery of a given limb. For example, the left arm cuff 102_{LA} shown in FIG. 5 is located approximately at the 3 o'clock to 4 o'clock position, which is configured to place the second sensor 146b adjacent to the brachial artery of the patient's left arm (with the assumed orientation of placing the arm into the cuff into/through the page of the drawing figure). If this were a right arm limb cuff 102_{RA}, the same sensor would be located approximately at the 8 o'clock to 9 o'clock position, to be adjacent to the brachial artery of the patient's right arm. If this were a leg limb cuff, the second sensor would be located in or on the inflatable sleeve adjacent to the patient's femoral artery. In the case of a left leg limb cuff 102_{LL}, this would be at approximately the 2 o'clock to 3 o'clock position and in the case of the right leg limb cuff 102_{RL}, this would be at approximately the 9 o'clock to 10 o'clock position.

In the alternative embodiment in which the real-time hemodynamic sensor consists of one single sensor that is configured for both proximal arterial waveform detection and measuring MLOP, that single real-time hemodynamic sensor would be located adjacent to the primary artery of a respective limb, i.e., at the location of the second sensor 146b as described herein. A microcontroller is located within the limb cuff 102 for providing autonomous operation of the real-time hemodynamic sensor 146 and the inflatable sleeve 106. Optionally, the microcontroller is an Arduino Nano or STM32.

The real-time hemodynamic sensor 146, which is optionally a digital ported pressure sensor such as the Adafruit MPRLS (3965), is configured to measure cuff pressure and arterial waveform characteristics with high temporal and spatial resolution. The MPRLS sensor is an absolute pressure device capable of measuring from 0 to 25 PSI with approximately ±0.02 PSI resolution. It contains an internal instrumentation amplifier, temperature compensation circuitry, and on-chip digital filtering. The sensor communicates with the microcontroller via an I²C digital interface, allowing stable, noise-resistant signal acquisition even in high-motion, artifact-prone environments such as during chest compressions.

The real-time hemodynamic sensor 146 is programmed to sample pressure continuously at a predetermined frequency (e.g., 50-100 Hz), and the microcontroller firmware that may be used applies real-time digital signal processing to extract hemodynamic information. This includes, for example: (i) determining and continuously updating MLOP, (ii) identifying proximal arterial pulsatility to assess perfusion, and (iii) filtering chest-compression artifacts by applying digital signal processing within the microcontroller, which subtracts acceleration-derived noise profiles obtained from onboard inertial sensors (e.g., an inertial measurement unit, IMU), from the raw pressure data acquired via the MPRLS sensor. The sensor's digital output allows the controller to perform waveform analysis, detect slope changes, and estimate blood flow using an Ohm's-law-derived algorithm in which flow is computed as the change in pressure (ΔP) divided by an estimated vascular resistance value.

In the illustrated embodiment, the first sensor 146a monitors intra-cuff pressure to maintain MLOP under dynamic CPR conditions, while the second sensor 146b detects proximal arterial waveform oscillations to determine perfusion status and ROSC. Upon recognition of ROSC, the microcontroller optionally initiates a programmed, gradual pressure-reduction sequence to maintain systemic vascular resistance while avoiding abrupt vascular redistribution. “Proximal arterial waveform detection” refers to the programming or the function of the second sensor 146b, which is not merely measuring static pressure. Rather, it is actively reading the arterial waveform, which is key for determining perfusion and cardiac output indicators during CPR. Optionally, the real-time hemodynamic sensor 146 (in the embodiment shown, the second sensor 146b) is configured to measure flow according to the following algorithm: flow=change in pressure divided by vascular resistance. This algorithm is based on Ohm's law. The “real time hemodynamic feedback” measured by the first sensor 146a provides actionable, immediate information that goes beyond traditional passive blood pressure monitoring. Rather, it measures MLOP in real-time, which changes as CPR is done. The first sensor 146a determines the minimal flow needed to preserve the limb while creating sufficient “traffic” at the point of occlusion for backflow to increase pressure in core of the patient's body.

When in use, there is a risk that vibrations or other movements could serve as “noise” that would interfere with sensor readings. Such movements could be due to chest compressions being administered to the patient during CPR and/or the patient being in transport (e.g., in an ambulance going over a bumpy road) on the way to a hospital. To address this issue, the limb cuff optionally includes an accelerometer 150, which acts to cancel such interference. Optionally, the accelerometer 150 is an LSM6DSO or similar inertial measurement unit. In the optional embodiment shown, the accelerometer 150 includes a first accelerometer component 150a located at about the 3 o'clock position of the housing 104 between the inner wall 110 and the external housing wall. This location is intended to position the first accelerometer component proximal to the patient's torso, from which some of the movement/interference would originate. The accelerometer 150 also includes a second accelerometer component 150 b located at about the 6 o'clock position of the housing between the inner wall 110 and the external housing wall. This location is intended to position the second accelerometer component 150b proximal to the ground or other substantially flat surface 12 upon which the limb cuff 102 rests, to cancel noise (e.g., vibrations) originating from that source.

In sum, as described above, each limb cuff 102 comprises: (a) an air pump 130 in fluid connection (preferably via tubing 134) with the inflatable sleeve 106, to inflate the inflatable sleeve 106 (to perform an occlusion operation); (b) a solenoid valve 142 in fluid connection with the inflatable sleeve 106 to release air from the inflatable sleeve 106 when the solenoid valve 142 is open; (c) a battery 132 (or other power source) to provide electrical power to the limb cuff 102; and (d) optionally an accelerometer 150 configured to cancel out external movement and noise to ensure accurate real-time hemodynamic readings.

Optionally, in any embodiment, there are no physical connections (e.g., tubing or wired electrical connections) between the plurality of limb cuffs 102 (aside from the fact that they are placed on the same patient). As such, each limb cuff 102 operates physically and electronically independently of every other limb cuff 102 when the system 100 comprises a plurality of limb cuffs 102. In other words, one limb cuff 102 does not directly communicate or direct the operation of the other limb cuffs, although the functioning of one limb cuff could have an indirect influence on the behavior of another limb cuff on account of the fact that they are placed on different limbs of the same patient. For example, if the right arm limb cuff 102_{RA} and left arm limb cuff 102_{LA} increase occlusion pressure, that would impact hemodynamic flow and pressure throughout the patient's circulatory system and thereby, perhaps indirectly influence the resultant behavior of the right leg cuff 102_{RL} and the right arm cuff 102_{LL}.

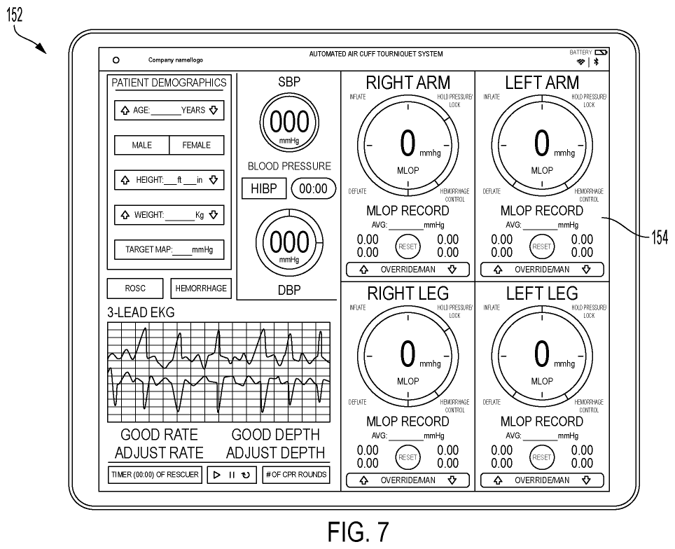

As shown in FIG. 7, the system 100 optionally includes a user interface 152 comprising a monitor 154 to display real time system status and patient hemodynamic feedback. The user interface 152 is physically separate from the plurality of limb cuffs 102 but is in wireless communication with them. Optionally, each limb cuff 102 comprises a wireless communication interface for remote monitoring and feedback. Optionally, the wireless communication interface transmits data via Bluetooth Low Energy (BLE), WiFi or Ultra Wideband between a respective limb cuff 102 and the user interface 152.

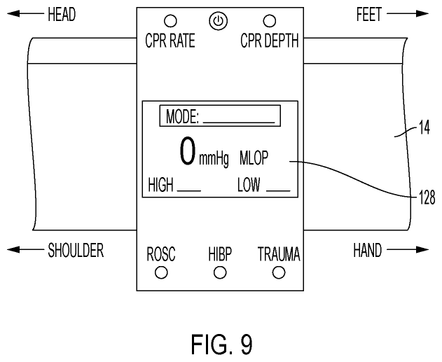

FIG. 9 shows an optional LCD screen 128 provided on the top of the housing 104 of a limb cuff 102. The optional LCD screen 128 is mounted on the top surface of the housing 104 and is configured to display real-time operational parameters of the limb cuff 102 to which it is affixed. The LCD screen 128 provides a clear visual interface for the user, showing critical indicators such as MLOP, mode of operation (e.g., CPR, ROSC, Hemorrhage Control, or High-Inflation Blood Pressure (HIBP)), and system status alerts. The digital display may include numerical readouts, bar indicators for occlusion pressure (e.g., LOW to HIGH), and mode-selection prompts for automated or manual override. Flanking the display, or integrated within the screen bezel, are LED indicators corresponding to CPR performance metrics: “CPR RATE” and “CPR DEPTH.” These LEDs provide immediate visual feedback based on real-time hemodynamic analysis (e.g., arterial waveform slope or pulsatility). Each parameter is illuminated using a color-coded system —red indicating poor performance, yellow indicating moderate, and green indicating optimal compression rate or depth. This allows rescuers to adjust compressions without relying on external monitoring devices, enhancing CPR quality assurance in real time. Additional tactile buttons or capacitive touch inputs may be provided adjacent to the display for rapid selection of operational modes in high-stress or low-resource environments. It should be noted that the foregoing description of the LCD screen 128 is merely exemplary and that various aspects may be omitted or combined with other display or input features not herein described.

### Methods of Using the Limb Cuffs and the System

In an optional aspect, the disclosed concept is directed to a method for augmenting blood supply to vital organs in a patient using (various optional embodiments of) the system 100 described herein, which utilizes the plurality of limb cuffs 102. With reference to FIGS. 1, 6A, 6B and 8, the method includes placing one limb cuff 102 on each separate limb 14 of the patient 10. The method further includes allowing each limb cuff 102 to autonomously perform a limb occlusion operation and to adjust a limb occlusion setting in response to real time hemodynamic readings generated by the real-time hemodynamic sensor so as to augment blood supply to the vital organs.

Optionally, this method may be used to treat a sudden medically destabilizing drop in blood pressure of the patient 10, for example due to sepsis or anaphylaxis.

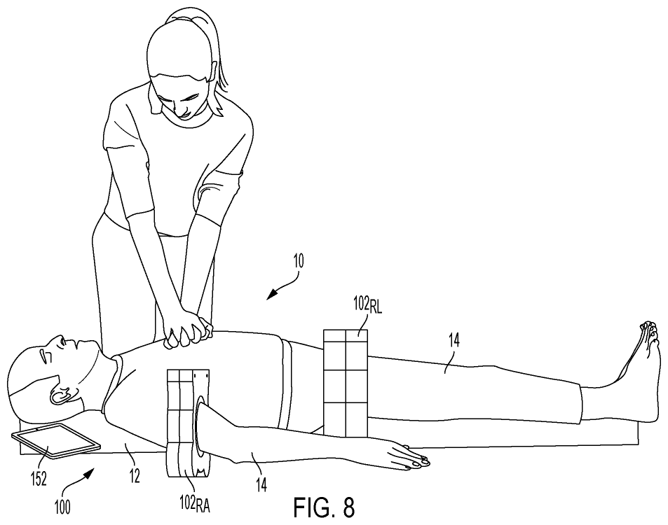

In addition or alternatively, the method may include any or all of the aforementioned steps and may be used for performing CPR on a patient. In such a case, the method would further include simultaneously applying chest compressions to the patient 10, e.g., as illustrated in FIG. 8. In this way, the system 100 would operate to increase blood supply to the vital organs during CPR, thereby helping to improve the likelihood of a good patient outcome. That is, by concentrating blood flow to vital organs through the occlusion operations performed by respective limb cuffs 102, the chance of preserving the patient's life increases substantially. Moreover, the real-time hemodynamic readings generated by the real-time hemodynamic sensor 146 autonomously control the amount of occlusion a given limb cuff 102 will provide (i.e., the extent of inflation of the inflatable sleeve 106 to provide a real-time calculated tourniquet pressure to the limb). The goal is not only to preserve the life of the patient, but to also preserve the limb by providing minimal blood circulation necessary to adequately sustain the limb while still applying necessary tourniquet pressure for perfusion of blood into vital organs during CPR.

Accordingly, when the system 100 is used during CPR, it determines MLOP (e.g. in mm Hg) of the patient, optionally using oscillometric feedback. The system 100 also determines blood flow at an occlusion site (adjacent to a primary limb artery) using hemodynamic feedback. A limb occlusion setting that corresponds to tourniquet pressure is automatically adjusted in real time in response to MLOP and blood flow measurements.

When using the system 100, preferably the leg limb cuffs are applied and activated before the arm limb cuffs. This is because there is much greater volume of blood flowing into the legs than the arms. In an acute situation therefore, occlusion of the legs should take first priority.

As the patient's condition changes during CPR—for example, if return of spontaneous circulation (ROSC) occurs—the system 100 can gradually reduce pressure through a ROSC-controlled release mode to maintain perfusion while preventing sudden blood redistribution. The goal is to optimize central blood pressure and organ perfusion without compromising limb safety. Thus, upon return of ROSC in the patient, an ROSC mode is initiated in the system 100 in which the plurality of limb cuffs 102 gradually reduce occlusion pressure, optionally in order to maintain stable systemic vascular resistance (SVR) and thereby mitigate a risk of acute central hypotension, which could lead to re-cardiac arrest and a need to restart CPR.

Optionally, in conjunction with use of the system 100, the patient is administered a vasopressor medication, optionally a member of the group selected from epinephrine, vasopressin, norepinephrine and phenylephrine. Alternatively, when the system 100 is used, it obviates the need for vasopressor medication and thus the patient is not administered such medication when carrying out methods of using the system 100.

Traditionally, chest compressions during CPR are performed by a human operator, e.g., as shown in FIG. 8. However, there are machines available to assist with or fully perform chest compressions. These include, for example, the LUCAS device by Stryker and AutoPulse by Zoll Medical. In the event such automated CPR devices are used, the system 100 would be compatible with them, i.e., the system 100 would not interfere with operation of such devices and vice versa. Optionally, the housing 104 can be augmented to fit, e.g., in a modular manner, other CPR supplementation (such as the LUCAS device).

Notably, the limb cuffs 102 of the system 100 are external devices, i.e., they do not involve insertion or implantation into the patient 10. Moreover, methods of using the system 100 described herein preferably do not increase intrathoracic pressure. Intrathoracic pressure is the pressure within the chest cavity and it is primarily influenced by ventilation mechanics. Devices that increase intrathoracic pressure directly affect preload, afterload and venous return to the heart; it is preferred that the limb cuffs 102 operate in a manner in which this does not happen. This preferred feature represents an advance over other methods, such as Resuscitative Endovascular Balloon Occlusion of the Aorta (REBOA), which increases intra-thoracic pressure.

It is contemplated that in some medical situations, use of only a single limb cuff 102 rather than a plurality may be appropriate. Such may be the case where the patient is not in cardiac arrest, but is hemorrhaging from a limb. In that case, the limb cuff 102 may be placed around the affected limb 14 at a location proximal to a site of hemorrhage in the limb 14. The limb cuff 102 is then allowed to autonomously perform a limb occlusion operation and to adjust a limb occlusion setting in response to the real time hemodynamic readings generated by the real-time hemodynamic sensor. This would, in turn, augment blood supply to vital organs, reduce hemorrhaging, and ensure MLOP and adequate blood flow at an occlusion site sufficient to substantially inhibit tissue decay in the limb due to insufficient blood flow. The limb occlusion setting is automatically adjusted in real time in response to MLOP and blood flow measurements. Accordingly, this method may be used to stop the hemorrhage while preserving life and the affected limb of the patient. Optionally, such hemorrhage control mode may effectuate a fixed occlusion pressure of from 200-300 mmHg) with optional periodic reperfusion for prolonged use.

As another optional feature, the system 100 could also function as an electrocardiography device (ECG or EKG). Electrodes for providing this function can be provided within the inflatable sleeves 106 of at least one of the limb cuffs 102 and in contact with the patient's skin when in use. Optionally, any or all of the four limb cuffs 102 can be used for this additional purpose. Leads I (electrical output direction from right arm to left arm), II (right arm to left leg) and III (left arm to left leg) may be found by the limb cuffs 102 that are adapted to include this feature.

In any embodiment, it is preferred that the limb cuffs 102 be placed “high and tight”. In the case of the arms, this means proximal to the armpits and in the case of the legs, proximal to the groin. This would enable the tourniquet to apply pressure as close to the torso as reasonably possible to concentrate blood flow in the torso and head, thereby aiding in effective perfusion.

### System Software and Settings

The real-time hemodynamic sensor 146 is configured for oscillometric sensing. Pressure data is sampled during chest compressions and filtered with low-pass and high-pass filters to extract arterial and compression waveforms.

MLOP is determined with an algorithm that relies on stepwise inflation with peak detection to identify maximal oscillation amplitude. Then, the inflatable sleeve is inflated to slightly above that pressure to achieve minimal limb occlusion pressure to avoid excessive limb ischemia.

CPR feedback (e.g., rate and depth) may be determined via an accelerometer.

ROSC mode enables the system 100 to recognize when ROSC follows CPR and allows for controlled, gradual deflation to begin upon such recognition of ROSC. ROSC mode may be initiated either manually (via button press) or automatically through physiologic indicators (e.g., oscillometric waveform changes, accelerometer shift, or EtCO₂ increase if applicable). Pressure is released incrementally to prevent sudden hypotension and promote hemodynamic stability. Optionally, the ROSC mode may be based on algorithmic estimation based on user inputs regarding patient demographics, e.g., age, sex, body mass index, limb circumference.

Optionally, the system 100 includes a lock function which locks MLOP during CPR and adjusts only if baseline pressures shift significantly.

### Exemplary Embodiments

The following exemplary embodiments further describe optional aspects of the presently disclosed technology and are part of this Detailed Description. These exemplary embodiments are set forth in a format substantially akin to claims (each with numerical designations followed by a capital letter), although they are not technically claims of the present application. The following exemplary embodiments refer to each other in dependent relationships as “embodiments” instead of “claims.”

1A. A system for augmenting blood supply to vital organs in a patient, optionally during cardiopulmonary resuscitation (CPR), the system comprising: a plurality of limb cuffs, each limb cuff of the plurality of limb cuffs comprising a real-time hemodynamic sensor and an inflatable sleeve configured to be placed around a limb of the patient to autonomously perform occlusion of the limb in response to real-time hemodynamic readings generated by the real-time hemodynamic sensor so as to augment blood supply to vital organs.

2A. The system of embodiment 1A, the inflatable sleeve having an inner surface configured to contact a respective limb while in use and an outer surface, each limb cuff comprising a housing that is more rigid than the inflatable sleeve, the housing comprising an inner wall having a fixed diameter that defines an outer boundary beyond which the inflatable sleeve cannot expand.

3A. The system of embodiment 1A, the housing comprising a bottom portion that is symmetrical and shaped in a manner that prevents rolling or rotation of the housing relative to a substantially flat surface on which the bottom portion is placed, optionally the ground, when the patient is lying in a supine position on the substantially flat surface and while the limb cuff is placed around a limb of the patient.

4A. The system of any one of embodiments 1A to 3A, wherein the plurality of limb cuffs consists of the following four limb cuffs:
a. a right arm limb cuff;
b. a left arm limb cuff;
c. a right leg limb cuff; and
d. a left leg limb cuff.

5A. The system of any one of embodiments 1A to 4A comprising a user interface comprising a monitor to provide real time system status and patient hemodynamic feedback, the user interface being physically separate from the plurality of limb cuffs.

6A. The system of any one of embodiments 1A to 5A, there being no physical connections between the plurality of limb cuffs.

7A. The system of any one of embodiments 1A to 6A, each limb cuff of the plurality of limb cuffs comprising a microcontroller for providing autonomous operation of the real-time hemodynamic sensor and the inflatable sleeve.

8A. The system of embodiment 7A, each limb cuff of the plurality of limb cuffs comprising:
a. an air pump in fluid connection with the inflatable sleeve to inflate the inflatable sleeve;
b. a solenoid valve in fluid connection with the inflatable sleeve to release air from the inflatable sleeve when the solenoid valve is open;
c. a power source, optionally a battery, to provide electrical power to each limb cuff; and
d. optionally an accelerometer configured to cancel out external movement and noise to ensure accurate real-time hemodynamic readings.

9A. The system of embodiment 8A, each limb cuff of the plurality of limb cuffs comprising a wireless communication interface for remote monitoring and feedback, optionally wherein the wireless communication interface transmits data via Bluetooth Low Energy (BLE), WiFi or Ultra Wideband to a user interface.

10A. The system of embodiment 8A or 9A, wherein the real-time hemodynamic sensor is configured to measure flow according to the following algorithm: flow=change in pressure divided by vascular resistance.

11A. The system of any one of embodiments 1A to 10A, wherein each limb cuff operates physically and electronically independently of every other limb cuff of the plurality of limb cuffs.

12A. The system of any one of embodiments 1A to 11A, wherein the real-time hemodynamic sensor comprises two of the same type of sensor, a first of which is configured for measuring minimal limb occlusion pressure and a second of which is configured for proximal arterial waveform detection.

1B. A method for augmenting blood supply to vital organs in a patient using the system of any one of embodiments 1A to 12A, the method comprising placing one limb cuff of the plurality of limb cuffs on each separate limb of the patient and allowing each limb cuff to autonomously perform a limb occlusion operation and to adjust a limb occlusion setting in response to the real time hemodynamic readings generated by the real-time hemodynamic sensor so as to augment blood supply to the vital organs.

2B. The method of embodiment 1B used to treat a sudden medically destabilizing drop in blood pressure of the patient, for example due to sepsis or anaphylaxis.

1C. A method for performing CPR on a patient comprising performing the method of embodiment 1B while simultaneously applying chest compressions to the patient.

2C. The method of embodiment 1C, wherein the system operates to increase blood supply to the vital organs during CPR.

3C. The method of embodiment 1C or 2C, wherein the system determines minimal limb occlusion pressure (MLOP, optionally in mm Hg) of the patient optionally using oscillometric feedback and also determines blood flow at an occlusion site using hemodynamic feedback, wherein a limb occlusion setting is automatically adjusted in real time in response to MLOP and blood flow measurements.

4C. The method of any one of embodiments 1C to 3C, wherein the plurality of limb cuffs consists of two lower extremity cuffs and two upper extremity cuffs, wherein each of the two lower extremity cuffs are placed on a respective leg of the patient and activated, after which each of the two upper extremity cuffs are placed on a respective arm of the patient and activated.

5C. The method of any one of embodiments 1C to 4C, wherein upon return of spontaneous circulation (ROSC) in the patient, an ROSC mode is initiated in the system in which the plurality of limb cuffs gradually reduce occlusion pressure, optionally in order to maintain stable systemic vascular resistance (SVR) and thereby mitigate a risk of acute central hypotension, which could lead to re-cardiac arrest.

1D. The method of any one of embodiments 1C to 5C, wherein the patient is administered a vasopressor medication, optionally a member of the group selected from epinephrine, vasopressin, norepinephrine and phenylephrine.

1E. The method of any one of embodiments 1C to 5C, wherein the patient is not administered a vasopressor medication.

1F. The method of any one of embodiments 1C to 5C, 1D or 1E, wherein the method is performed while an automated mechanical CPR device (e.g., LUCAS device by Stryker or AutoPulse by Zoll Medical) applies the chest compressions to the patient.

2F. The method of any one of embodiments 1C to 5C, 1D or 1E, wherein the method is compatible to be performed as an automated mechanical CPR device (e.g., LUCAS device by Stryker or AutoPulse by Zoll Medical) applies the chest compressions to the patient.

3F. The method of any one of embodiments 1C to 5C, 1D, 1E, 1F or 2F, wherein the plurality of limb cuffs are external devices (i.e., do not involve insertion or implantation into the patient) and the method does not increase intrathoracic pressure.

1G. A method for controlling hemorrhage from a limb of a patient, the method comprising placing a limb cuff around the limb at a location proximal to a site of hemorrhage in the limb, the limb cuff comprising a real-time hemodynamic sensor and an inflatable sleeve, the method further comprising allowing the limb cuff to autonomously perform a limb occlusion operation and to adjust a limb occlusion setting in response to the real time hemodynamic readings generated by the real-time hemodynamic sensor so as to augment blood supply to vital organs and to ensure minimal limb occlusion pressure (MLOP, optionally in mm Hg) and adequate blood flow at an occlusion site sufficient to substantially inhibit tissue decay in the limb due to insufficient blood flow, wherein a limb occlusion setting is automatically adjusted in real time in response to MLOP and blood flow measurements.

While specific embodiments of the invention have been described in detail, it will be appreciated by those skilled in the art that various modifications and alternatives to those details could be developed in light of the overall teachings of the disclosure. Accordingly, the particular arrangements disclosed are meant to be illustrative only and not limiting as to the scope of disclosed concept which is to be given the full breadth of the claims appended and any and all equivalents thereof.

What is claimed is:

1. A method for augmenting blood supply to vital organs in a patient, the method comprising:
a. providing a system comprising a plurality of limb cuffs, each limb cuff of the plurality of limb cuffs comprising a real-time hemodynamic sensor and an inflatable sleeve configured to be placed around a limb of the patient to autonomously perform occlusion of the limb in response to real-time hemodynamic readings generated by the real-time hemodynamic sensor; and
b. placing one limb cuff of the plurality of limb cuffs on each separate limb of the patient and allowing each limb cuff to autonomously perform a limb occlusion operation and to adjust a limb occlusion setting in response to the real time hemodynamic readings generated by the real-time hemodynamic sensor so as to augment blood supply to the vital organs.

2. The method of claim 1, the inflatable sleeve having an inner surface configured to contact a respective limb while in use and an outer surface, each limb cuff comprising a housing that is more rigid than the inflatable sleeve, the housing comprising an inner wall having a fixed diameter that defines an outer boundary beyond which the inflatable sleeve cannot expand.

3. The method of any previous claim, the housing comprising a bottom portion that is symmetrical and shaped in a manner that prevents rolling or rotation of the housing relative to a substantially flat surface on which the bottom portion is placed, optionally the ground, when the patient is lying in a supine position on the substantially flat surface and while the limb cuff is placed around a limb of the patient.

4. The method of any previous claim, wherein the plurality of limb cuffs consists of the following four limb cuffs:
a. a right arm limb cuff;
b. a left arm limb cuff;
c. a right leg limb cuff; and
d. a left leg limb cuff.

5. The method of any previous claim, the system comprising a user interface comprising a monitor to provide real time system status and patient hemodynamic feedback, the user interface being physically separate from the plurality of limb cuffs.

6. The method of any previous claim, there being no physical connections between the plurality of limb cuffs.

7. The method of claim 6, there being no wires or tubes connecting the plurality of limb cuffs.

8. The method of any previous claim, each limb cuff of the plurality of limb cuffs comprising a microcontroller for providing autonomous operation of the real-time hemodynamic sensor and the inflatable sleeve.

9. The method of any previous claim, each limb cuff of the plurality of limb cuffs comprising:
a. an air pump in fluid connection with the inflatable sleeve to inflate the inflatable sleeve;
b. a solenoid valve in fluid connection with the inflatable sleeve to release air from the inflatable sleeve when the solenoid valve is open;
c. a power source, optionally a battery, to provide electrical power to each limb cuff; and
d. optionally an accelerometer configured to cancel out external movement and noise to ensure accurate real-time hemodynamic readings.

10. The method of any previous claim, each limb cuff of the plurality of limb cuffs comprising a wireless communication interface for remote monitoring and feedback, optionally wherein the wireless communication interface transmits data via Bluetooth Low Energy (BLE), WiFi or Ultra Wideband to a user interface.

11. The method of any previous claim, wherein the real-time hemodynamic sensor is configured to measure flow according to the following algorithm: flow =change in pressure divided by vascular resistance.

12. The method of any previous claim, wherein each limb cuff operates physically and electronically independently of every other limb cuff of the plurality of limb cuffs.

13. The method of any previous claim, wherein the real-time hemodynamic sensor comprises two of the same type of sensor, a first of which is configured for measuring minimal limb occlusion pressure (MLOP) and a second of which is configured for proximal arterial waveform detection.

14. A method of performing cardiopulmonary resuscitation (CPR) on a patient comprising performing the method of any previous claim while simultaneously applying chest compressions to the patient.

15. The method of claim 14, wherein the system operates to increase blood supply to the vital organs during CPR.

16. The method of claim 14 or 15, wherein the system determines minimal limb occlusion pressure (MLOP, optionally in mm Hg) of the patient optionally using oscillometric feedback and also determines blood flow at an occlusion site using hemodynamic feedback, wherein a limb occlusion setting is automatically adjusted in real time in response to MLOP and blood flow measurements.

17. The method of any one of claims 14 to 16, wherein the plurality of limb cuffs consists of two lower extremity cuffs and two upper extremity cuffs, wherein each of the two lower extremity cuffs are placed on a respective leg of the patient and activated, after which each of the two upper extremity cuffs are placed on a respective arm of the patient and activated.

18. The method of any one of claims 14 to 17, wherein upon return of spontaneous circulation (ROSC) in the patient, an ROSC mode is initiated in the system in which the plurality of limb cuffs gradually reduce occlusion pressure in order to maintain stable systemic vascular resistance (SVR) and thereby mitigate a risk of acute central hypotension.

19. The method of any one of claims 14 to 18, wherein the method is capable of being performed while an automated mechanical CPR device applies the chest compressions to the patient.

20. The method of any previous claim, wherein the method does not increase intrathoracic pressure.
